(19) United States
(12) Patent Application Publication
(10) Pub. No.: US 2026/0261634 A1
(43) Pub. Date: Sep. 3, 2026

(54) PROJECTOR, CONNECTED OBJECT MANAGER, DIRECT NEURAL INTERFACE, CONNECTED ELECTRONIC EQUIPMENT, PROJECTOR INITIALISATION METHOD AND METHOD FOR CONTROLLING ELECTRONIC EQUIPMENT

(71) Applicant: ORANGE, Issy-les-Moulineaux (FR)

(72) Inventors: Cyril Plapous, Chatillon (FR); Foued Bouchnak, Chatillon (FR)

(21) Appl. No.: 18/866,235

(22) PCT Filed: May 15, 2023
(86) PCT No.: PCT/EP2023/062875

§ 371 Date: Nov. 15, 2024

(30) Foreign Application Priority Data
May 17, 2022 (FR) 2204684

(51) Int. Cl. H04N 9/31; G06F 3/01

(52) U.S. Cl. CPC H04N 9/3173; G06F 3/015

(57) ABSTRACT

A projector of a luminous flux with a given frequency, a connected object manager, a direct neural interface, electronic equipment connected to the communication network, a projector initialization method and a method for controlling connected electronic equipment, and in particular, an interaction with electronic equipment via a direct neural interface. A projector of a luminous flux with a given frequency in a given projection zone is anchored on electronic equipment connected to a communication network. The projector is connected to a communication network. The given frequency is associated with a given command of the electronic equipment. Thus, the projector makes it possible to add neural stimulation to incompatible electronic equipment, in particular equipment not provided with a screen, available to several users.

### TECHNICAL FIELD

The invention relates to a projector of streams of light with a given frequency, to a manager of connected objects, to a direct neural interface, to an electronic device connected to the communication network, to a projector-initialization method and to a method of control of a connected electronic device. Particularly, the invention relates to interaction with an electronic device by means of a direct neural interface.

### PRIOR ART

Direct neural interfaces have been used to allow accessibility to electronic devices to be expanded to more people, and particularly to people with physical disabilities.

Currently, electronic devices must be equipped with a display to reproduce a specific stimulus. This stimulus triggers appearance of a particular signal, also called the visual evoked potential, in the subject's EEG signals (EEG standing for electroencephalogram) when she or he receives visual stimulation with this stimulus. A specific device, for example a BCI headset (BCI standing for brain computer interface), i.e. a direct neural interface-headset allowing EEG signals to be measured, detects in these EEG signals a frequency that corresponds to the frequency of the visual stimulus. Virtual and/or augmented reality headsets are further beginning to employ direct neural interfaces.

Thus, a greater number of users, including users without disabilities, will be in a position to use a direct neural interface to facilitate and simplify interaction with electronic devices, thus reducing errors made thereby during interactions with devices.

However, direct neural interfaces make it possible to interact only with certain electronic devices, also known as BCI-compatible electronic devices, which are manufactured specifically to be connected to a direct neural interface, such as a BCI headset, particularly in order to increase the accessibility of such BCI-compatible electronic devices. In particular, these BCI-compatible electronic devices must comprise a display allowing visual stimuli to be reproduced with a given frequency, and a controller capable of controlling the electronic device depending on the frequency received from the direct neural interface.

Electronic devices that are not BCI-compatible cannot be controlled by means of a direct neural interface, making use thereof more complex for everyone (thus leading to errors during interaction), or even impossible for some people. Particularly, electronic devices which are able to be controlled by one or more physical buttons (whatever the type of physical button: push button, touch button, etc.) require the user to be able to manipulate these buttons correctly, which she or he may be prevented from doing, either by her or his disability, or because her or his hands are constrained either by a garment (gloves for example) or by an object. Because of this impediment, the device may trigger an erroneous action, either because the user pressed the wrong button, or because the user did not press any button following a request by the device.

One solution is to add via augmented reality neural-interaction signals to devices that are not BCI-compatible. The drawback of augmented reality is that it limits the neural interaction to one user, the one wearing the augmented-reality glasses.

### SUMMARY OF THE INVENTION

One of the aims of the present invention is to rectify drawbacks of the prior art.

One subject of the invention is a projector of a stream of light with a given frequency into a given projection area anchored to an electronic device that is connected to a communication network, the projector being connected to a communication network, the given frequency being associated with a given command of the electronic device.

Thus, the projector allows incompatible electronic devices, in particular electronic devices without a display, to be endowed with a neural-stimulation capability, which capability is accessible to a plurality of users.

Advantageously, the given command is identical to a given command triggered by a physical button of the electronic device.

Thus, a given command of an electronic device may be actuated both via a physical button and by means of neural interaction associated with the stream of light emitted by the projector.

Advantageously, the projector comprises a recognizer of the electronic device, this recognizer delivering an electronic-device identifier depending on which the given frequency is determined.

Thus, the projector may be used to produce a neural stimulation for a plurality of distinct electronic devices and not just one, in particular during movement of a user wearing the projector or for a plurality of electronic devices in the same room, the devices for example lying in the area covered by the light emitted by the projector.

Advantageously, the projector comprises a locator for locating the electronic device, the locator delivering an electronic-device position depending on which the given projection area is determined.

Thus, a neural interaction remains possible with a plurality of distinct electronic devices and/or when the projector is nomadic.

Advantageously, the projector emits a plurality of streams of light with distinct given frequencies.

Thus, a neural interaction allowing more than one distinct given commands to be generated for one or more electronic devices is possible.

Advantageously, the given frequencies are associated with given commands of a plurality of electronic devices, each stream of light being projected with a given frequency into the given projection area around the electronic device a given command of which is associated with the given frequency among predefined geographic areas around the electronic devices.

Thus, neural-interaction errors are reduced since the area of projection by the projector emitting the visual stimulus (the stream of light) for the direct neural interaction, is positionally associated with the electronic device to be controlled. Therefore, a user looking at the electronic device with which she or he wishes to interact therefore perceives, at the same time, the stream of light emitted by the projector, this triggering an EEG signal with a neural frequency corresponding to the given frequency of the stream of light emitted by the projector, which frequency is itself associated with the given command of the physical button.

Advantageously, the projector is one of the following devices:
a component of a direct neural interface capable of sensing a user-related frequency during projection of the stream of light with the given frequency, the direct neural interface comprising a sensed-neural-frequency transmitter capable of triggering a given command of the electronic device, which given command is associated with the given frequency of the stream of light emitted by the projector, the transmitted neural frequency being dependent on the given frequency;
a fixed projector comprising an attachment to a fixed device relative to at least one electronic device;
a nomadic projector comprising an attachment to a user portable device.

Another subject of the invention is a manager of objects connected to a communication network, which objects include at least a projector and an electronic device, wherein the manager of connected objects comprises a coupler capable of associating a given frequency of a stream of light emitted by a projector into a given projection area anchored to an electronic device connected to the communication network with a given command of the electronic device connected to the communication network.

Yet another subject of the invention is a manager of objects connected to a communication network, which objects include at least a projector and an electronic device, wherein the manager of connected objects comprises a controller capable of controlling, by means of a given command, an electronic device connected to the communication network, depending on a neural frequency received from a direct neural interface during emission of a stream of light with a given frequency by the projector into a given projection area anchored to an electronic device connected to the communication network, the received neural frequency being dependent on the given frequency, the given frequency and the given command being associated.

Advantageously, the manager of connected objects comprises the coupler and the controller.

Another subject of the invention is a direct neural interface capable of sensing a user-related frequency during projection of a stream of light with a given frequency, wherein the direct neural interface comprises a sensed-neural-frequency transmitter that transmits via a communication network, the sensed-neural-frequency transmitter being capable of triggering a given command of an electronic device associated with the given frequency of a stream of light emitted by a projector into a given projection area anchored to an electronic device connected to the communication network, the transmitted neural frequency being dependent on the given frequency.

Another subject of the invention is an electronic device connected to a communication network, wherein the electronic device comprises an actuator capable of controlling a component of the electronic device depending on at least one given command, the given command being dependent on a neural frequency transmitted by a direct neural interface during emission of a stream of light with a given frequency by a projector into a given projection area anchored to an electronic device connected to the communication network, the transmitted neural frequency being dependent on the given frequency, the given frequency and the given command being associated.

Another subject of the invention is a method of initialization of a projector and of an electronic device that is connected to a communication network, wherein the initialization method comprises an association associating a given frequency of a stream of light emitted by a projector into a given projection area anchored to an electronic device connected to the communication network with a given command of an electronic device connected to the communication network.

A further subject of the invention is a method of control of an electronic device, wherein the control method controls, by means of a given command, an electronic device connected to a communication network depending on a neural frequency received from a direct neural interface during emission of a stream of light with a given frequency by a projector into a given projection area anchored to an electronic device connected to the communication network, the received neural frequency being dependent on the given frequency, the given frequency and the given command being associated.

Advantageously, according to one implementation of the invention, the various steps of the method according to the invention are implemented by software or a computer program, this software comprising software instructions intended to be executed by a data processor of a device forming part of a manager of connected objects and being designed to control execution of the various steps of this method.

The invention therefore also relates to a program comprising program code instructions for executing the steps of the method of initialization of a projector and/or of the method of control of an electronic device when said program is executed by a processor.

This program may use any programming language, and take the form of source code, object code or intermediate code between source code and object code, such as in a partially compiled form or in any other desirable form.

### BRIEF DESCRIPTION OF THE DRAWINGS

The features and advantages of the invention will become more clearly apparent on reading the description, which is given by way of example, and the related figures, in which:

FIG. 1 shows a simplified schematic of an architecture comprising a neural stimulator-projector and an electronic device according to the invention,

FIG. 2 shows a simplified schematic of an architecture comprising a manager of connected objects including a neural stimulator-projector and an electronic device according to the invention,

FIG. 3 shows a simplified schematic of exchanges during implementation of a method of initialization of a neural stimulator-projector according to the invention,

FIG. 4 shows a simplified schematic of exchanges during implementation of a method of control of an electronic device using a neural stimulator-projector according to the invention,

FIG. 5a shows a simplified schematic of a first mode of use of a neural stimulator-projector according to the invention, in which the neural stimulator-projector is placed on an electronic device,

FIG. 5b shows a simplified schematic of a second mode of use of a neural stimulator-projector according to the invention, in which the neural stimulator-projector projects onto a plurality of electronic devices placed in a room,

FIG. 5c shows a simplified schematic of a third mode of use of a neural stimulator-projector according to the invention, in which the neural stimulator-projector is attached to, or even integrated into, the direct neural interface worn by the user.

### DESCRIPTION OF THE EMBODIMENTS

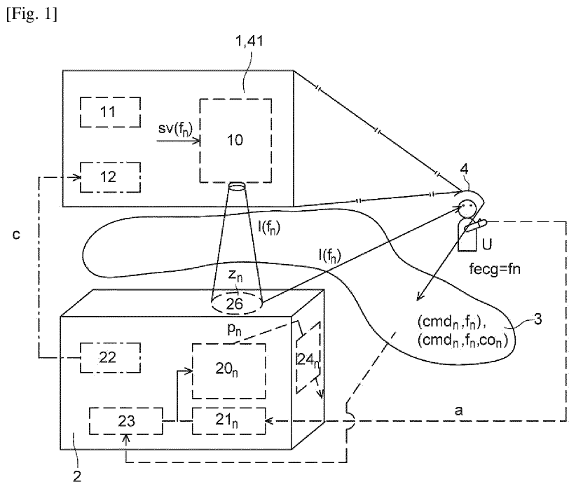

FIG. 1 illustrates a simplified diagram of an architecture comprising a neural stimulator-projector and an electronic device according to the invention.

The projector 1 emits a stream of light l(fn) with a given frequency fn into a given projection area zₙ, 26ₙ anchored to an electronic device 2 connected to the communication network 3. The projector 1 is connected to the communication network 3. The given frequency fn is associated with a given command cmdₙ of the electronic device 2.

Particularly, the given projection area zₙ is also referenced 26ₙ when it is on the electronic device 2 to be controlled by means of a given command cmdₙ depending on the given frequency fn with which the stream of light l(fn) is projected into this given projection area zₙ, 26ₙ.

Particularly, the projector 1 comprises an emitter 10 capable of emitting a stream of light l(fn) carrying a visual signal sv(fn) with the given frequency fn. The visual projecting device 10 is in particular a 2D projector, a 3D projector, a holographic projector, etc. and/or a targeted projector, a 360° projector, etc. The projector 1 emits a stream of light at least one pixel of which varies, and in particular flickers, at at least one given frequency.

Particularly, the projector 1, in particular the emitter 10, emits a visual stimulus l(fn) with the given frequency corresponding to reproduction of the visual signal sv(fn) with the given frequency in the form of the stream of light l(fn). The visual stimulus l(fn) is perceptible, i.e. visible, to a user U.

Particularly, the given command is identical to a given command triggered by a physical button 21n of the electronic device.

Particularly, the given projection area zₙ, 26ₙ is associated positionally, i.e. for example in terms of position and location, with the physical button 21n, and in particular anchored to the physical button 21n. For example, if the electronic device is a lamp, the given projection area will be associated with the on/off switch of this lamp.

By positional association, what is meant is the fact that the given projection area is placed, in terms of position, in proximity to the physical button or to the sign of the physical button with which it is associated. By positional proximity, what is meant is the fact that the given projection area is placed directly on the physical button, or even at a distance from the associated physical button less than the distance to the physical button of the electronic device closest to the associated physical button, or indeed, if the electronic device is equipped with a plurality of devices placed on the same straight line, on a straight line perpendicular to this straight line passing through the associated physical button, etc.

Thus, BCI interaction errors are reduced since the projection area into which the projector emits the visual stimulus with a view to direct neural interaction is positionally associated with the physical button of the interaction to be triggered. Therefore, a user looking at the button with which she or he wishes to interact therefore perceives, at the same time, the stream of light emitted by the projector into the positionally associated given projection area, this triggering an EEG signal with a frequency corresponding to the given frequency of the stream of light emitted by the projector, which frequency is itself associated with the given command of the physical button.

Since a conventional physical button must in particular be pressed physically or detect when it is actuated, the projector according to the invention adds to the existing button an ability to generate visual stimuli, particularly luminous visual stimuli, in particular through emission of such a visual stimulus. Since the visual stimulus may be detected in the EEG signals by means of a direct neural interface, for example one implemented in a BCI headset, the invention thus allows a user to interact with the electronic device as though she or he were interacting with the conventional physical button through an additional interaction modality. Thus, the user may actuate the physical button without pressing it, or even touching it, only using EEG signals.

A physical button 21n is defined in contrast to a virtual button. The physical button 21n is in particular a push button, a touch button, an optical button, etc. The physical button 21n allows one or more commands to be triggered. In the case of a multi-command physical button, the multi-command button is in particular:
a toggle switch for switching between a plurality of contactors. For example, the toggle switch toggles between two positions triggering two distinct and potentially contrary commands: activation/deactivation of a component of the electronic device, or is a D-pad offering four positions triggering four distinct commands of the same type, in particular commands to make different movements: forward movement/backward movement/movement to the right/movement to the left, etc.;
a rotary switch for switching between a plurality of different command positions: in particular various predefined values of a given command: for example temperature, volume, etc. or a plurality of operating modes of the electronic device, and/or of a component of the electronic device, such as a different operating mode of a smart oven: grill, fan oven, etc., or activation of a component of this smart oven: switching on its light, updating the clock, etc.

A distinct frequency will be associated with each position of the multi-command button. A first stream with a first frequency is emitted into a given first projection area z₁ associated with a first position of the multi-command button: the projector 1 will emit a first stream of light l1(f1) with a first given frequency f1 distinct from the second given frequency f2 used by the projector 1 to emit a second stream of light l2(f2) into a second given projection area z₂ associated with a second position of the multi-command button. The first given projection area and the second given projection area, and the first position of the multi-command button and the second position of the associated multi-command button, respectively, are distinct.

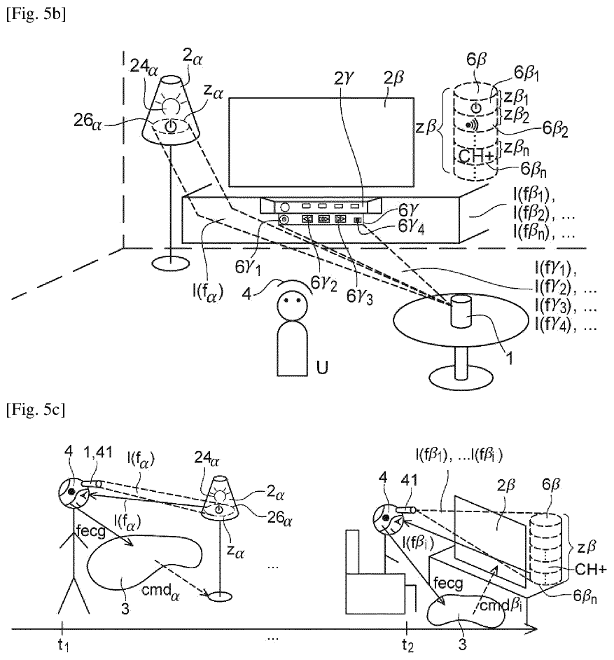

Alternatively, the given projection area zₙ, 26ₙ is associated positionally, i.e. for example in terms of position and location, with the component 24n of the electronic device 2 controlled by the given command fn, and in particular anchored to this component 24n. For example, if the electronic device 2 is a lamp 2_{α}, the given projection area z_{α} will be associated with the bulb 24_{α} of this lamp 2_{α} (see the examples of FIGS. 5b and 5c).

Particularly, the projector comprises a recognizer of the electronic device, this recognizer delivering an electronic-device identifier depending on which the given frequency is determined.

In particular, the recognizer is an identifier receiver able to communicate directly with the electronic device with a view to receiving an identifier of the electronic device, for example via a local network such as a Bluetooth network, an NFC network, etc.

Optionally, the recognizer comprises a sensor and recognizing means that are capable of sensing data relating to the electronic device: image, sound, input and/or output data flow, etc. and of achieving identification, through recognition and in particular by means of artificial intelligence, through learning or through comparison with data recorded in a recognition database. Optionally, the recognizer determines an electronic-device category to which the identified electronic device belongs.

Thus, the recognizer makes it possible to determine commands capable of being implemented by the electronic device, among which commands at least one command will be selected to be associated with the given frequency: the given command.

Particularly, the projector comprises a locator of the electronic device, the locator delivering an electronic-device position depending on which the given projection area is determined.

Particularly, the projector emits a plurality of streams of light with distinct given frequencies.

Particularly, the given frequencies are associated with given commands of a plurality of electronic devices, each stream of light being projected with a given frequency into the given projection area around the electronic device a given command of which is associated with the given frequency among predefined geographic areas around the electronic devices.

Particularly, the projector 1 is one of the following devices:
a component of a direct neural interface capable of sensing a user-related frequency during projection of the stream of light with the given frequency, the direct neural interface comprising a sensed-neural-frequency transmitter capable of triggering a given command of the electronic device, which given command is associated with the given frequency of the stream of light emitted by the projector, the transmitted neural frequency being dependent on the given frequency;
a fixed projector comprising an attachment 11 to a fixed device relative to at least one electronic device;
a nomadic projector comprising an attachment 11 to a user portable device.

In the case of a fixed projector 1, the attachment 11 in particular comprises a rotary platform on which the emitter 11 is placed so that a stream of light may be emitted toward a plurality of devices in the same room. In one alternative embodiment, the emitter 11 allows 360° emission of the one or more streams of light at given frequency.

Particularly, when the projector 1 is a targeted projector, i.e. one that solely emits a stream of light in a given projection area or streams of light in contiguous given areas, the attachment 11 of the fixed projector 1 is capable of holding the projector 1 in relation to the electronic device 2, in particular in relation to the physical button 21n (see the example in FIG. 5a).

Thus, the given projection area of the stream of light cannot move, limiting interaction errors related to loss of the positional association of the given projection area with the physical button the given command of which is associated with the given frequency fn of the stream of light l(fn) emitted by the projector.

In the case of a nomadic projector 1, the attachment 11 in particular allows the projector to be attached to a direct neural interface 4, and in particular to a BCI headset, to an augmented-reality headset, to a device worn by the user (such as glasses, an earpiece, a cap, etc.), or even directly to the user (the attachment is then a shoulder pad, a spectacle frame, a headband and/or armband, etc.

Advantageously, the projector 1 comprises a rechargeable battery 12. In particular, the projector 1 may be connected in respect of supply of power to any electronic device, such as the electronic device 2, a computer, a tablet, etc. in particular by means of a USB cable, with a view to being recharged, or even to an electrical socket via a battery charger.

The electronic device 2 is connected to a communication network 3. The electronic device 2 comprises an actuator 20n capable of controlling a component 24n of the electronic device depending on at least one given command, the given command being dependent on a neural frequency transmitted by a direct neural interface during emission of a stream of light with a given frequency by a projector into a given projection area anchored to an electronic device connected to the communication network, the transmitted neural frequency being dependent on the given frequency, the given frequency and the given command being associated.

In particular, the actuator 20n is a processor 20n capable of executing processing depending on at least one command.

Particularly, the electronic device 2 comprises a power supply 22 for the projector 1 when the projector 1 is placed on, against or in proximity to the electronic device 2, in particular when the projector 1 is fixed in place in relation to the electronic device 2.

Particularly, the power supply 22 implemented in the electronic device 2 recharges c the battery 12 of the projector 1, in particular when the projector 1 is connected to the electronic device 2, for example, by a cable, via induction, etc.

Particularly, the power supply of the projector 22 is one of the following power supplies:
a power-supply connector capable of being connected to the projector when the projector area of the projector is associated with a physical button of the electronic device;
an induction-based charger capable of recharging a battery of the projector when the projection area of the projector is associated with a physical button of the electronic device.

Particularly, the projector 1 and the electronic device 2 are two distinct devices in that the projector 1 and the electronic device 2 are separate. By projector 1 distinct from the electronic device 2, what is in particular meant is the fact that the electronic device 2 does not comprise and does not implement the projector 1.

The direct neural interface 4 is capable of sensing a frequency f_{ecg} related to a user U during projection of a stream of light l(fn) with a given frequency fn. The direct neural interface 4 comprises a transmitter 44 (not illustrated) for transmitting the sensed neural frequency f_{ecg} via a communication network 3. The sensed-neural-frequency transmitter is capable of triggering a given command cmdₙ of an electronic device 2 associated with the given frequency of a stream of light l(fn) emitted into a given projection area zₙ, 26ₙ anchored to an electronic device 2 connected to the communication network 3. The transmitted neural frequency f_{ecg} is dependent on the given frequency fn.

Particularly, the transmitter 44 of the direct neural interface 4 is a communication interface that is able to communicate with the communication network 3.

Particularly, since the given frequency fn is associated with a given command cmdₙ of the electronic device 2, the sensed neural frequency f_{ecg} corresponding to the given frequency fn of the visual signal sv(fn) reproduced by the emission of the stream of light l(fn) looked at by the user U is therefore associated with this same command cmdₙ of the electronic device 2. The sensed neural frequency f_{ecg} is therefore capable of controlling, via cmdₙ, the electronic device.

Particularly, the transmitter 44 is capable of delivering the sensed neural frequency f_{ecg} related to a user U sensed during emission of a stream of light l(fn) with a given frequency fn by a projector connected to a communication network 3. The sensed neural frequency f_{ecg} is a frequency of a visual-evoked-potential signal in the electroencephalogram signals of a user U.

A given frequency fn of emission of a stream of light by a projector connected to a communication network 3 is associated with a given command cmdₙ of an electronic device 2 connected to the communication network 3. The projector 1 and the electronic device 2 are distinct.

Optionally, the projection area zₙ, 26ₙ of the stream of light emitted by the projector 1 is positionally associated with a physical button 21n of the electronic device 2. A manipulation of the physical button triggers the given command cmdₙ.

Particularly, the emitter 10 of the projector 1 emits a stream of light with the given frequency l(fn) allowing a visual signal to be reproduced with the given frequency sv(fn).

When the user U looks at a visual stimulus l(fn) resulting from projection of the stream of light l(fn) with the given frequency fn by the projector 1, a direct neural interface 4 senses a neural frequency f_{ecg} related to the user U. The sensed neural frequency f_{ecg} is dependent on the given frequency fn—in particular they have the same value.

Particularly, the direct neural interface 4 is implemented in a BCI headset or neural headset or even in an augmented-reality headset which then possesses EEG capabilities, i.e. is able to detect electroencephalogram signals.

The direct neural interface 4 delivers the sensed neural frequency f_{ecg} via the communication network 3. A given command cmdₙ dependent on the sensed neural frequency is implemented by the electronic device 2.

Optionally, the electronic device 2 receives from an analyzer (not illustrated in FIG. 1) depending on the neural frequency emitted by the direct neural interface 4 and on the pair, or even triplet, associating a given command and a given frequency (cmdₙ,fₙ), (cmdₙ,fn,zₙ), (cmdₙ,fn,coₙ) the given command cmdₙ. The analyzer is in particular implemented in a device of the communication network 3 distinct from the projector 1 and/or the electronic device 2 and/or the direct neural interface 4.

The direct neural interface 4 implementing a projector thus allows one or more given projection areas to be easily associated with a given electronic device but also projection areas to be associated with a high number of electronic devices. This increases possibilities in respect of equipping electronic devices (smart sockets, smart switches, Hi-Fi's, smart speakers, domestic router, TV decoder, DVD player, smart appliances, remote control, keyboard, ‘stop’ button for stopping a bus, etc.) with means for interacting neurally, but above all in respect of the number of electronic devices that are equipped with means for interacting neurally in a given place, such as a home, an office, etc. with a plurality of users, in particular in a nomadic mode, i.e. when the user equipped with the direct neural interface including a projector is able to move about.

Particularly, the projector 1 is connected to the communication network 3 via a local wireless network such as Wi-Fi network, Bluetooth network, LoRa network, etc.

The projector 1 is therefore a neural stimulator. Therefore, the projector 1 is also called the neural stimulator-projector 1. The neural stimulator-projector 1 comprises an emitter 10 of a stream of light with the given frequency l(fn).

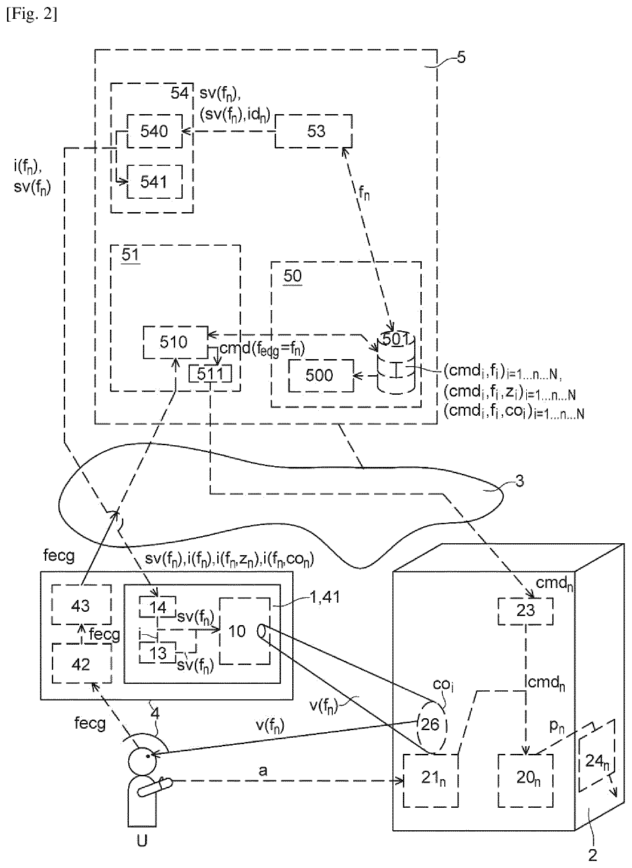

FIG. 2 illustrates a simplified schematic of an architecture comprising a manager of connected objects including a neural stimulator-projector and an electronic device according to the invention,

The manager manages objects connected to a communication network, these objects including at least a projector and an electronic device.

By object, what is meant is any solid thing, considered as a whole, that is manufactured and that is intended for a certain use. A connected object is then an object capable of exchanging a signal via a domestic communication network (via a wired LAN or a wireless LAN: Wi-Fi LAN, Bluetooth LAN, LoRA LAN, etc.) and/or a WAN such as wired IP WAN (ADSL, fiber, etc.) or wireless (4G, 5G, etc.) IP WAN, with at least one electronic device such as a device for delivering services.

By connected object, what is meant is a physical or virtual object interconnected via at least one local-area and/or wide-area communication network with a view to delivering advanced services by virtue of information and/or communication technologies.

The communication network 3 is a wide-area network (WAN) or a domestic, local-area network (LAN).

Particularly, the projector 1 is connected to a network of the type referred to by the acronym IoT (standing for Internet of Things) managed by the manager 5 of connected objects. The IoT network is in particular associated with a specific location, such as the user's home, a workshop and/or a building of a company, etc.

Particularly, the manager 5 of connected objects comprises a coupler 50 capable of associating a given frequency fn of a stream of light l(fn) emitted into a given projection area zₙ, 26ₙ anchored to an electronic device 2 connected to the communication network 3 with a given command cmdₙ of the electronic device 2. Optionally, the coupler 50 is capable of associating the given frequency fn, the given area zₙ (in particular the location of the given area) and the given command cmdₙ.

Particularly, the coupler 50 comprises a storage device 501 for storing pairs of given commands—given frequencies (cmdᵢ, fi)_{i=1 . . . n . . . N}. The storage device 501 is a memory, a command database, etc.

Particularly, the coupler 50 comprises a recorder 500 capable of storing the given command—given frequency pair (cmdₙ, fₙ), in particular in a storage device 501 of the manager 50 of connected objects.

Particularly, when the projector 1 is a nomadic projector and/or a 360° projector, the coupler 50 associates the given frequency fn, the given command cmdₙ and a given projection area zₙ, 26ₙ. The given projection area zn is in particular defined by coordinates coₙ (including, for example, the 2D or 3D position of the center of the projection area and/or a radius of coverage of the projection area, or even the 2D or 3D position of a point on the periphery of the projection area and/or one or more dimensions of this periphery).

In this case, the pairs are replaced by triplets of given commands—given frequencies—given projection areas (cmdᵢ, fi, coᵢ)_{i=1 . . . n . . . N}. Thus, the optional storage device 501 is a storage device 501 for storing triplets of given commands—given frequencies—given projection areas (cmdᵢ, fi, coᵢ)_{i=1 . . . n . . . N}, and the potential recorder 500 is able to store the triplet of given commands—given frequencies—given projection areas (cmdᵢ, fi, coᵢ)_{i=1 . . . n . . . N}.

Particularly, the projector 1 comprises a modifier of the beam of a stream of light, which is capable of modifying a direction and/or a width of a beam of the stream of light emitted with a given frequency l(fn), in such a way that the beam of the stream of light with a given frequency covers only the given projection area zₙ, 26ₙ. The modifier is in particular composed of mirrors and/or reflectors, and/or of a motor allowing the emitter 10 to be rotated.

Particularly, the manager 5 of connected objects comprises a controller 51 capable of controlling, by means of a given command cmdₙ, an electronic device 2 connected to the communication network 3 depending on a neural frequency f_{ecg} received from a direct neural interface 4 during emission of a stream of light l(fn) with a given frequency fn by the projector 1 into a given projection area zₙ, 26ₙ anchored to the electronic device 2. The received neural frequency f_{ecg} is dependent on the given frequency fn. The given frequency fn and the given command cmdₙ are associated.

Particularly, the controller 51 is capable of controlling an electronic device 2 connected to the communication network 3 depending on a neural frequency f_{ecg} received from a direct neural interface 4 when the user U of the direct neural interface 4 performs an action related to emission of a stream of light l(fn) with a given frequency fn by the projector 1. The action performed by the user U is in particular to look at the visual stimulus resulting from emission of a stream of light l(fn) with the given frequency fn.

Particularly, the controller 51 comprises an analyzer 510 capable of determining a given command cmdₙ of the electronic device 2 depending on a neural frequency f_{ecg} received from a direct neural interface 4 during emission by the projector 1 of a stream of light l(fn) with the given frequency fn associated with the given command cmdₙ.

Particularly, the analyzer 510 requests from the storage device 501 the given command cmdₙ associated with a given frequency fn corresponding to the received neural frequency f_{ecg} and delivers the given command cmdₙ to the electronic device 2.

Particularly, the controller 51 comprises a transmitter 511 of commands to the electronic device 2, the command transmitter transmitting a given command cmdₙ associated with a given frequency fn corresponding to the received neural frequency f_{ecg}.

Particularly, the analyzer 510 delivers the given command cmdₙ to the command transmitter 511.

Particularly, the manager 5 of connected objects transmits the given command cmdₙ to the electronic device over an MQTT bus (MQTT standing for Message Queuing Telemetry Transport) in particular using a Wi-Fi protocol, particularly when the manager 5 of connected objects is remote from the electronic device 2.

Optionally, the controller 51 comprises a trigger 511 capable of initiating implementation and/or execution, by the electronic device 2, of a given command cmdₙ associated with a given frequency fn corresponding to the received neural frequency f_{ecg}:cmdₙ(f_{ecg}=fn).

In a first architecture (not illustrated), a first manager 5′ of connected objects comprises the coupler 50′ and a second manager 5″ of connected objects comprises the controller 51″.

Alternatively, in a second architecture illustrated in FIG. 2, a manager 5 of connected objects comprises the coupler 50 and the controller 51. Particularly, the manager 5 of connected objects comprises:
the coupler 50 of given frequency fn of a stream of light l(fn) emitted by the projector 1 with a given command cmdₙ of an electronic device 2, and
the controller 51 of connected devices depending on a neural frequency f_{ecg} received from a direct neural interface 4 during emission of a stream of light l(fn) at the given frequency fn by the projector 1.

Particularly, the manager 5 of connected objects comprises a transceiver 54 that exchanges a signal i(fn), sv(fn) relating to the given frequency fn with the projector 1.

Particularly, the signal relating to the given frequency fn is one of the following signals:
a signal i(fn) indicative of the given frequency fn and/or of the coordinates coₙ of the given projection area zₙ, 26ₙ transmitted by the manager 5 of connected objects to the projector 1;
a visual signal sv(fn) to be emitted with the given frequency fn, which signal is generated by the manager 5 of connected objects and transmitted by the manager 5 of connected objects to the projector 1;
a signal i(fn) indicative of the given frequency fn transmitted by the projector 1 to the manager 5 of connected objects.

Particularly, the transceiver 54 comprises a transmitter 540 of the signal relating to the given frequency fn to the projector 1.

Particularly, the transceiver 54 comprises a receiver 540 of the indicative signal i(fn) delivered by the projector 1.

Particularly, the manager 5 of connected objects comprises a generator 53 of a visual signal sv(fn) with a given frequency fn. The visual signal thus generated sv(fn) is capable of being reproduced. In particular, the generator 53 delivers the generated visual signal sv(fn), optionally with an identifier idn of the destination projector, to the transceiver 54, and particularly to the transmitter 540.

A projector 1 is connected to a communication network 3 capable of emitting a stream of light l(fn) with a given frequency fn. Particularly, the stream of light allows a visual signal sv(fn) to be reproduced. Optionally, the given frequency fn is associated by the manager 5 of connected objects with a given command cmdₙ of an electronic device 2 connected to the communication network 3. The projector 1 is distinct from the electronic device 2.

Thus, an electronic device 2 that does not have a display or any other type of visual reproduction device is able to be endowed with a so-called BCI function, i.e. a neural interface, by means of the projector and the manager of connected objects according to the invention.

Particularly, the projector 1 comprises an emitter 10 capable of emitting a stream of light l(fn) with the given frequency fn. The emitter 10 is in particular a 2D projector, a 3D projector, a holographic projector, etc. and/or a targeted projector or a 360° projector, etc.

The projector 1, and particularly the emitter 10, emits a visual stimulus l(fn) with the given frequency allowing reproduction of a visual signal sv(fn) with the given frequency. The visual stimulus l(fn) is perceptible, i.e. visible, to a user U.

The projector 1 in particular comprises a receiver 14 capable of receiving:
either the signal i(fn) indicative of the given frequency fn transmitted by the manager 5 of connected objects to the projector 1;
or the visual signal sv(fn) to be reproduced with the given frequency fn, which signal is generated by the manager 5 of connected objects and transmitted by the manager 5 of connected objects to the projector 1.

Particularly, the projector 1 comprises a generator 13 of a visual signal sv(fn) with a given frequency fn, for example depending on the given frequency fn received with the signal i(fn) indicative of the given frequency fn delivered by the object manager 5. The visual signal thus generated sv(fn) is capable of being transmitted in the form of a stream of light l(fn) by the projector 1. In particular, the generator 13 delivers the generated visual signal sv(fn), potentially to the emitter 10.

Particularly, the projector 1 and the electronic device 2 are two distinct devices in that the projector 1 and the electronic device 2 are separate. By projector 1 distinct from the electronic device 2, what is in particular meant is the fact that the electronic device 2 does not comprise and does not implement the projector 1.

Particularly, the given command cmdₙ is identical to a given command triggered by a physical button 21n of the electronic device 2. The projection area zₙ, 26ₙ of the projector 1 is positionally associated ph_ass with the physical button 21n.

By positional association, what is meant is the fact that the given area zₙ, 26ₙ of projection of the stream l(fn) with a given frequency by the projector 1 is placed, in terms of position and location, in proximity to the physical button or to the sign of the physical button with which it is associated. By positional proximity, what is meant is the fact that the given area zₙ, 26ₙ of projection of the stream l(fn) with a given frequency by the projector 1 is placed directly on the physical button, or even at a distance from the associated physical button less than the distance to the physical button of the electronic device closest to the associated physical button, or indeed, if the electronic device is equipped with a plurality of buttons placed on the same straight line, on a straight line perpendicular to this straight line passing through the associated physical button, etc.

Thus, BCI interaction errors are reduced since the given area zₙ, 26ₙ of projection of the stream l(fn) with a given frequency by the projector 1 reproducing the visual stimulus with a view to direct neural interaction is positionally associated with the physical button of the interaction to be triggered. Therefore, the user looking at the button with which she or he wishes to interact therefore perceives, at the same time, the stream of light l(fn) emitted by the projector 1, this triggering an EEG signal with a frequency corresponding to the given frequency fn of the stream of light l(fn) emitted by the projector 1, which frequency is itself associated with the given command of the physical button.

A physical button 21n is in particular a button such as defined with regard to FIG. 1.

An electronic device 2 connected to a communication network 3 comprises an actuator 20n capable of controlling a component of the electronic device 2 depending on at least one given command cmdₙ, for example delivered by a manager 5 of connected objects. The given command c cmdₙ is dependent on a neural frequency f_{ecg} transmitted by a direct neural interface 4 during emission of a stream of light l(fn) with a given frequency fn by a projector 1. The transmitted neural frequency f_{ecg} is dependent on the given frequency fn.

In particular, the actuator 20n is a processor 20n capable of executing processing depending on at least one command.

Particularly, the electronic device 2 comprises a control interface 24 capable of receiving a given command from a manager 5 of connected objects via the communication network 3 depending on a neural frequency f_{ecg} received from a direct neural interface 4 during reproduction of a visual signal sv(fn) with a given frequency fn by the projector 1. The received neural frequency f_{ecg} is dependent on the given frequency fn.

Particularly, the electronic device 2 comprises a power supply of the projector 22 when the projector 1 is attached, or even in proximity, to the electronic device 2.

Particularly, the power supply 22 implemented in the electronic device 2 recharges the battery 12 of the projector 1, in particular when the projector 1 is connected to the electronic device 2, for example, by a cable, via induction, etc.

Particularly, the power supply of the projector 22 is one of the following power supplies:
a power-supply connector capable of being connected to the projector 1 when the projector 1 is associated with a physical button of the electronic device;
an induction-based charger capable of recharging a battery of the projector 1 when the projector 1 is associated with a physical button of the electronic device.

A direct neural interface 4 is capable of sensing a frequency f_{ecg} related to a user U during emission of a stream of light l(fn) with a given frequency fn. The direct neural interface 4 comprises a transmitter 44 (not illustrated) of the sensed neural frequency f_{ecg} to a manager of objects that are connected via a communication network 3. The sensed-neural-frequency transmitter is capable of triggering a given command cmdₙ of an electronic device 2 associated with the given frequency fn of a stream of light l(fn) emitted by a projector 1 via the communication network 3. The transmitted neural frequency f_{ecg} is dependent on the given frequency fn.

Particularly, the direct neural interface 4 is capable of being connected to a manager 5 of connected objects.

Particularly, a direct neural interface 4 is capable of delivering a neural frequency f_{ecg} sensed during emission of a stream of light l(fn) with a given frequency fn. The direct neural interface 4 comprises a communication interface 44 capable of being connected to a manager 5 of connected objects. The communication interface 44 is capable of transmitting, to the manager 5 of connected objects, the neural frequency f_{ecg} sensed during emission of a stream of light l(fn) with a given frequency fn by the projector 1.

Particularly, the transmitter 44 is a communication interface that is able to communicate with the communication network 3 connected to the manager 5 of connected objects.

Particularly, the given frequency fn being associated, by the manager 5 of connected objects, with a given command cmdₙ of the electronic device 2, the sensed neural frequency f_{ecg} corresponding to the given frequency fn of the emitted stream of light looked at by the user U is therefore associated with this same command cmdₙ of the electronic device 2. The sensed neural frequency is therefore capable of controlling, via cmdₙ, the electronic device.

Particularly, the transmitter 44 is capable of delivering the neural frequency f_{ecg} related to a user U sensed during emission of a stream of light l(fn) with a given frequency fn by a projector 1 connected to a communication network 3.

The sensed neural frequency f_{ecg} is a frequency of a visual-evoked-potential signal in the electroencephalogram signals of a user U.

A given frequency fn of emission of a stream of light by a projector 1 connected to a communication network 3 is associated, by the manager 5 of connected objects, with a given command cmdₙ of an electronic device 2 connected to the communication network 3. The projector 1 and the electronic device are distinct.

Optionally, the projector 1 is positionally associated ph_ass with a physical button 21n of the electronic device. A manipulation of the physical button triggers the given command cmdₙ.

Particularly, in parallel with the positional association ph_ass, the manager 5 of connected objects associates, in particular by means of a coupler 50, the given command cmdₙ corresponding to the physical button 21n with a given frequency fn usable by the projector 1 to emit a stream of light l(fn), in particular depending on a visual signal sv(fn).

Particularly, the manager 5 of connected objects transmits, in particular by means of a transceiver 54 and/or more particularly of a transmitter 540, to the projector 1, the visual signal with the given frequency sv(fn) or an indicative signal i, i(fn), i(fn,zₙ), i(fn,coₙ). The visual signal with the given frequency sv(fn) transmitted by the manager 5 of connected objects is optionally generated by the manager 5 of connected objects, and in particular by a visual-signal generator 53.

Particularly, the emitter 10 of the projector 1 emits a stream of light l(fn) with the given frequency depending on the visual signal with the given frequency sv(fn) into a given projection area zₙ, 26ₙ, in particular after receipt of the visual signal with the given frequency sv(fn) or an indicative signal i, i(fn), i(fn,zₙ), i(fn,coₙ), in particular by means of a receiver 14. Optionally, the projector 1, particularly by means of a visual-signal generator 13, generates the visual signal with the given frequency sv(fn) depending on the signal indicative of the given frequency i, i(fn), i(fn,zₙ), i(fn,coₙ), which indicative signal is received from the manager of connected objects.

Either the projector 1 is a fixed targeted projector and the given projection area zₙ, 26ₙ is then defined when the projector 1 is positioned relative to the electronic device 2, or even relative to the physical button 21n.

Or the projector 1 is a 360° projector and/or a nomadic projector and the given projection area zₙ, 26ₙ is then controlled by the emitter 10 and/or a motor (not illustrated) for moving, and in particular rotating, the emitter, etc. depending on given projection coordinates coₙ associated with the given frequency fn and with the given command cmdₙ that are optionally delivered by the manager 5 of connected objects, in particular via an indicative signal i(fn,zₙ), i(fn,coₙ).

When the user U looks at a visual stimulus resulting from emission of the stream of light l(fn) with the given frequency fn by the projector 1, a direct neural interface 4 senses a neural frequency f_{ecg} related to the user U. The sensed neural frequency f_{ecg} and the given frequency fn have the same value.

Particularly, the direct neural interface 4 is implemented in a BCI headset or even an augmented-reality headset.

The direct neural interface 4 delivers, in particular by means of a transmitter 44, the sensed neural frequency f_{ecg} via the communication network 3, particularly to the manager 5 of connected objects. A given command cmdₙ dependent on the sensed neural frequency is implemented by the electronic device 2.

Optionally, the electronic device 2 receives, from a controller 51, and particularly from an analyzer 510, in particular implemented in the manager 5 of connected objects, depending on the neural frequency transmitted by the direct neural interface 4 and on the pair associating a given command and a given frequency (cmdₙ,fₙ), the given command cmdₙ.

Particularly, a communication terminal of the user comprises a configuration interface (not illustrated) for configuring the manager 5 of connected objects. This configuration interface allows the user to command the coupler 50 of the manager of connected objects to associate a given command cmdₙ of the electronic device 2 with a given frequency fn of the projector 1, or even a given projection area zₙ, 26ₙ of the projector. The configuration interface is in particular implemented on the communication terminal in the form of an application.

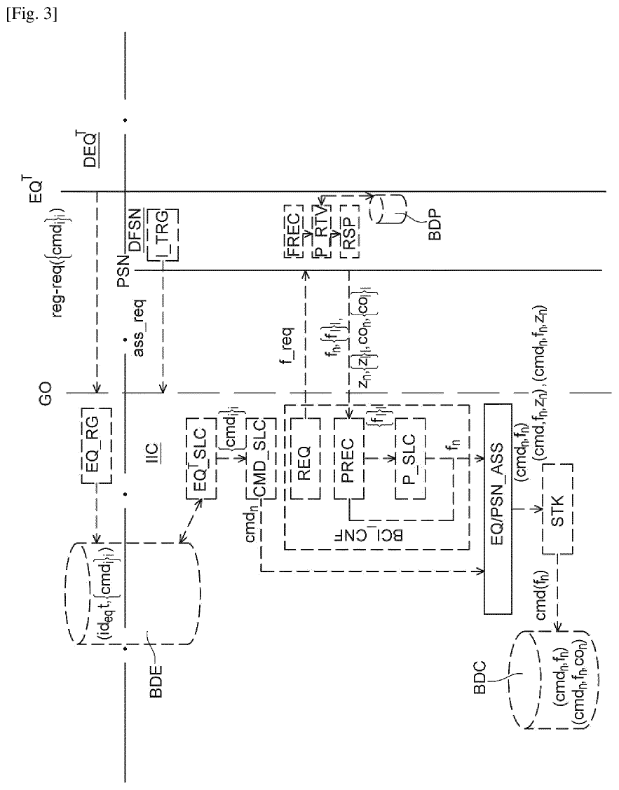

FIG. 3 illustrates a simplified schematic of exchanges during implementation of a method of initialization of a neural stimulator-projector according to the invention.

The initialization method IIC allows a projector PSN and an electronic device EQt that is connected to a communication network to be initialized. The initialization method IIC comprises an association EQ/PSN_ASS associating a given frequency fn of a stream of light l(fn) emitted by a projector PSN into a given projection area zₙ, 26ₙ anchored to an electronic device EQt connected to the communication network with a given command cmdₙ of an electronic device EQt connected to the communication network.

Particularly, the initialization method IIC comprises, prior to the association EQ/PSN_ASS, a selection EQt_SLC of a connected electronic device in particular from a plurality of connected devices in particular managed by a manager GO of connected objects. The electronic-device selection EQt_SLC in particular delivers the commands {cmdi}i that are capable of being implemented by the selected electronic device EQt. Optionally, the electronic-device selection EQt_SLC retrieves the list of commands {cmdᵢ}ᵢ capable of being implemented by the selected electronic device EQt either from the selected electronic device EQt (option not illustrated) or from a storage device, in particular a database BDE of electronic devices that is optionally stored in the manager GO of connected objects.

Particularly, the initialization method IIC comprises, prior to the association EQ/PSN_ASS, a selection CMD_SLC of a command of an electronic device, particularly from a list of commands {cmdᵢ}ᵢ of a previously selected electronic device EQt_SLC. In the case where a connected neural stimulator-interface IC is physically associated with a physical button of the electronic device EQt, the selected command cmdₙ, also known as the given command, corresponds to the command triggered by an action of a user U on the physical button of the electronic device EQt.

Particularly, the initialization method IIC comprises, prior to the association EQ/PSN_ASS, a neural configuration BCI_CNF delivering a given frequency fn, or even a given frequency fn and a given projection area zₙ potentially defined by coordinates coₙ, which is/are suitable for use by the projector PSN in the process of being associated with the electronic device EQt.

Particularly, the initialization method IIC comprises, prior to the association EQ/PSN_ASS, a reception of parameters PREC whereby the following is received from the projector PSN: either a list of frequencies {fl]ₗ suitable for being used by the projector PSN to emit streams of light, or the given frequency fn, or lists of given frequencies {fl]ₗ and of given projection areas {zₗ]ₗ, {coₗ]ₗ suitable for being used by the projector PSN to emit streams of light, or the given frequency fn and the given projection area zₙ, coₙ. Particularly, the neural configuration BCI_CNF comprises the reception of parameters PREC.

Particularly, the initialization method IIC comprises, prior to the frequency reception FREC, a frequency request REQ that sends and possibly generates before sending it a frequency request f_req to the connected neural stimulator-interface IC. Particularly, the neural configuration BCI_CNF comprises the frequency request REQ prior to the frequency reception FREC.

Particularly, the initialization method IIC comprises, prior to the association EQ/PSN_ASS, a selection P_SLC of given parameters, in particular of a given frequency fn among the frequencies {fl]ₗ suitable for being used by the projector PSN in the process of being associated with the electronic device EQt, and, where appropriate, of a given projection area zₙ, zₙ(coₙ) among the projection areas {zₗ]ₗ into which the projector PSN in the process of being associated with the electronic device EQt is capable of emitting. Particularly, the neural configuration BCI_CNF comprises the selection of given parameters P_SLC.

Particularly, the initialization method IIC comprises a storage STCK of the given command-given frequency pair (cmdₙ, fn), or even of the given command-given frequency-given projection area triplet (cmdₙ,fn,zₙ), (cmdₙ,fn,coₙ), delivered by the association EQ/PSN_ASS. The storage STCK records the given command-given frequency pair (cmdₙ, fn), or even the given command-given frequency-given projection area triplet (cmdₙ, fn, zₙ), (cmdₙ, fn, coₙ), in a storage device, in particular a database BDC of neural commands.

Particularly, the initialization method IIC is triggered by an association request ass_req passed from the projector PSN to the electronic device EQt. Particularly, the projector PSN actuates an initialization trigger I_TRG that causes the association request ass_req to be sent.

Particularly, a method DPSN of declaration of the projector is implemented by the projector PSN. The method DPSN of declaration of the projector comprises the initialization trigger __ TRG.

Optionally, the method DPSN of declaration of the projector comprises a parameter declaration RSP that sends, to the initialization method, and in particular to the frequency reception FREC, either a list of frequencies {fl]ₗ suitable for being used by the projector PSN to emit streams of light, or the given frequency fn, or lists of given frequencies {fl]ₗ and of given projection areas {zₗ]ₗ, {coₗ]ₗ suitable for being used by the projector PSN to emit streams of light, or the given frequency fn and the given projection area zₙ, coₙ.

Particularly, the method DPSN of declaration of the projector comprises, prior to the parameter declaration RSP, a search P_RTV for parameters usable by the projector PSN, and in particular for one or more usable frequencies, and where appropriate for one or more usable projection areas (in particular defined by projection-area coordinates). The parameter search P_RTV reads the usable parameters (frequencies, projection areas) recorded beforehand in a storage device, in particular one or more databases BDP of parameters (or even a database of frequencies and, where appropriate, a database of projection areas) optionally implemented in the projector PSN.

Particularly, the method DPSN of declaration of the projector comprises, prior to the parameter declaration RSP and, where appropriate, the parameter search P_RTV, reception of a parameter request FREC originating from the initialization method IIC.

In one particular embodiment, prior to the initialization method IIC, an electronic device EQt implements a device declaration method DEQt. The device declaration method DEQt sends, via the communication network to which it is connected, a registration request reg_req to a device registration method EQ_REG in particular implemented by the manager GO of connected objects. The registration request in particular contains a list of the commands {cmdᵢ}i capable of being implemented by the electronic device EQt.: reg_req({cmdᵢ}i).

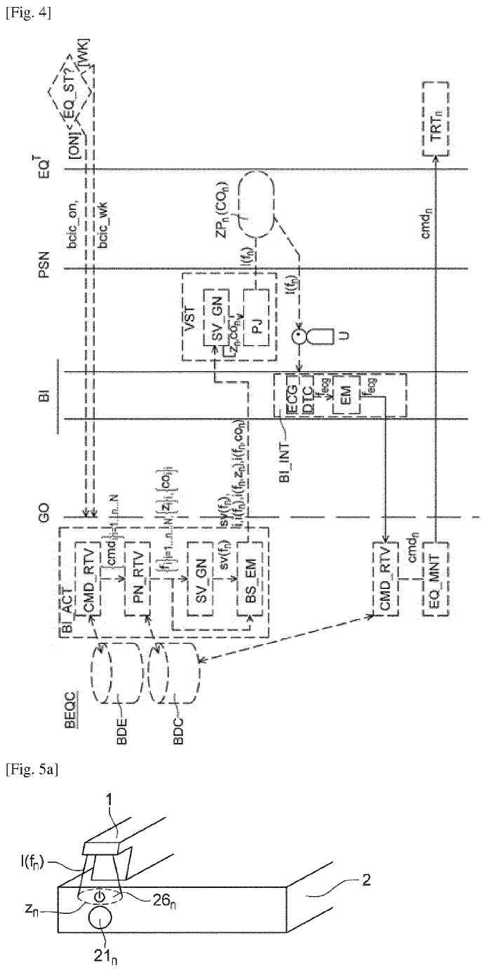

FIG. 4 illustrates a simplified schematic of exchanges during implementation of a method of control of an electronic device using a neural stimulator-projector according to the invention,

The method BEQC of control of an electronic device controls, by means of a given command cmdₙ, an electronic device EQt connected to a communication network depending on a neural frequency f_{ecg} received from a direct neural interface BI during emission of a stream of light l(fₙ) with a given frequency fn by a projector PSN into a given projection area ZPn anchored to an electronic device EQt connected to the communication network. The received neural frequency f_{ecg} is dependent on the given frequency fn. The given frequency fn and the given command cmdₙ are associated.

Particularly, the control method BEQC is implemented by a manager GO of connected objects.

Particularly, the control method BEQC comprises an activation BI_ACT of an emission PJ of a stream of light l(fn) with a given frequency fn, in particular implemented by a projector PSN. A visual stimulation method VST, in particular implemented by the projector PSN, comprises emission PJ of the stream of light l(fn) with a given frequency fn.

Particularly, the projector PSN emits PJ the stream of light l(fn) with a given frequency fn in a given projection area ZPn, in particular defined by projection-area coordinates coₙ.

Particularly, the activation BI_ACT of emission of a stream of light with a given frequency l(fn) comprises a neural-stimulation emitter BS_EM emitting a signal sv(fn), i, i(fn), i(fn,zₙ), i(fn,coₙ).

Particularly, the trigger BS_EM sends a signal relating to the given frequency fn used during emission PJ of a stream of light with the given frequency fn. The signal sent by the trigger BS_EM is in particular:
either an indicative signal i, such as a signal i indicative of the given frequency fn and/or of the given projection area zₙ: i, i(fn), i(fn,zₙ), i(fn,coₙ), sent to the emission PJ and/or to the visual stimulation method VST;
or a visual signal sv(fn) to be reproduced with the given frequency fn, sent to the emission PJ and/or to the visual stimulation method VST.

Particularly, the visual stimulation method VST comprises, prior to the emission PJ, a generation SV_GN of a visual signal sv(fn) with a given frequency fn indicated in the indicative signal i(fn), i(fn,zₙ), i(fn,coₙ) delivered by the trigger BS_EM.

Particularly, the electronic-device control method BEQC comprises, prior to the trigger BS_EM of emission of the stream of light with a given frequency l(fn), a search PN_RTV for the given parameters associated with the commands of the electronic device EQt, i.e. for the given frequencies and, where appropriate, the given projection areas. Particularly, the search for given parameters PN_RTV, in particular in a storage device such as a database BDC of commands, for example the database of commands of FIG. 3.

Particularly, the electronic-device control method BEQC comprises, prior to the trigger BS_EM of emission of a stream of light with a given frequency l(fn), a generation SV_GN of a visual signal sv(fn) with a given frequency fn indicated in the indicative signal i(fn), i(fn,zₙ), i(fn,coₙ) delivered by the trigger BS_EM.

Particularly, the electronic-device control method BECQ comprises, prior to the search PN_RTV for given parameters, a search for commands of the electronic device CMD_RTV, in particular in a storage device such as a database BDE of electronic devices, for example the database of electronic devices of FIG. 3, which delivers a list of commands {cmdᵢ}_{i=1 . . . n . . . N} capable of being implemented by the electronic device EQt.

Optionally, the search for commands CMD_RTV is triggered by an activation command bcic_on or a wake-up command bcic_wk of one or more connected neural stimulator-interfaces ICn associated with the electronic device EQt.

For example, a check EQ_ST? of the state of the electronic device determines whether the device is changing from an “off” or “deactivated” state to an “on” or “activated” state.

When a change from the “off” or “deactivated” state to an “on” or “activated” state is detected [ON] by this check EQ_ST?, then the check EQ_ST? sends an activation command bcic_on. The activation command bcic_on optionally involves listing commands, delivering a list of commands {cmdᵢ}_{i=1 . . . n . . . N} capable of being implemented by the electronic device EQt. The activation command bcic_on triggers activation BI_ACT of emission PJ of a stream of light l(fn) with a given frequency fn and/or the search CMD_RTV for commands.

When a change from the “standby” state to an “on” or “activated” state is detected [WK] by this check EQ_ST?, then the check EQ_ST? sends a wake-up command bcic_wk. The wake-up command bcic_wk optionally involves listing commands, delivering a list of commands {cmdᵢ}_{i=1 . . . n . . . N} capable of being implemented by the electronic device EQt. The wake-up command bcic_wk triggers activation BI ACT of emission PJ of a stream of light l(fn) with a given frequency fn and/or the search CMD_RTV for commands.

Particularly, the electronic-device control method BEQC comprises, after trigger BS_EM of emission PJ of a stream of light with a given frequency l(fn), a supervision EQ_MNT of the electronic device, which controls the electronic device EQt by means of a given command cmdₙ dependent on a sensed neural frequency f_{ecg} delivered by a direct neural interface BI during emission PJ of the stream of light with a given frequency l(fn) by a projector PSN combined with the electronic device EQt.

Particularly, the electronic-device control method BEQC comprises, after trigger BS_EM of emission PJ of a stream of light with a given frequency l(fn) and prior to the given command cmdₙ of the electronic device EQt, in particular to transmission of the given command cmdₙ by the supervision EQ_MNT of the electronic device, a search CMDN_RTV for given commands depending on a sensed neural frequency f_{ecg} delivered by a direct neural interface BI during emission PJ of the stream of light with a given frequency l(fn) by a projector PSN associated with the electronic device EQt. The search CMDN_RTV for a given command is performed in a storage device such as the database BDC of commands, for example the database of commands of FIG. 3.

Optionally, the emission PJ of the stream of light with a given frequency l(fn) is controlled in terms of emission direction and/or the width or angle of the emission beam via parameters of the given projection area zₙ, in particular via coordinates of a given projection area coₙ defining the given projection area ZPn.

Particularly, a neural-interaction method BI_INT delivers the sensed neural frequency f_{ecg} delivered by a direct neural interface BI during emission PJ of the stream of light with a given frequency l(fn) by a projector PSN associated with the electronic device EQt.

The neural-interaction method BI_INT is in particular implemented by the direct neural interface BI.

Particularly, a neural frequency detection ECG_DTC delivers, during emission PJ of the stream of light with a given frequency l(fn) by a projector PSN associated with the electronic device EQt, the sensed neural frequency. The neural frequency detection is in particular activated by an action of the user U of the direct neural interface BI, such as the user U looking at the visual stimulus v(fn) resulting from emission PJ of a stream of light with a given frequency l(fn).

Particularly, the neural-interaction method BI_INT comprises neural frequency detection ECG_DTC.

Particularly, a sensed neural frequency transmission EM transmits, to the electronic-device control method BEQC, the neural frequency f_{ecg} sensed during emission PJ of the stream of light with a given frequency l(fn) by a projector PSN associated with the electronic device EQt. In particular, the sensed neural frequency f_{ecg} transmitted by the transmission EM is delivered by the neural frequency detection ECG_DTC.

Particularly, the neural-interaction method BI_INT comprises the sensed neural frequency transmission EM.

Particularly, the given command cmdₙ triggers execution of given processing TRTn by the electronic device EQt.

In one particular embodiment, a method OCMNGT of management of connected objects comprises a method IIC of initialization of a connected neural stimulator-interface and a method BEQC of control of electronic devices. For a specific electronic device, the management method OCMNGT implements the method IIC of initialization of at least one connected neural stimulator-interface associated with this specific electronic device prior to implementation of the control method BEQC for this specific electronic device.

In one embodiment of the method IIC of initialization of a connected neural stimulator-interface and/or the method BEQC of control of an electronic device and/or the method OCMNGT of management of connected objects is a program comprising program code instructions for executing the steps of the method of initialization of a connected neural stimulator-interface and/or of the method of control of an electronic device and/or of the method of management of connected objects when said program is executed by a processor.

In one particular embodiment, the initialization method and/or the control method are/is implemented by at least one program comprising program code instructions for executing the steps of the method of initialization of a projector and/or of the method of control of an electronic device when said program is executed by a processor.

FIGS. 5a to 5c illustrate various modes of use of a neural stimulator-projector according to.

FIG. 5a shows a simplified schematic of a first mode of use of a neural stimulator-projector according to the invention, in which the neural stimulator-projector is placed on an electronic device.

The projector 1 is a fixed targeted projector. The projector 1 is placed on the electronic device 2 to which a neural-interaction function is added. In the example in FIG. 5a, the electronic device is an HDD and/or DVD media player, or an HDD and/or DVD media player/recorder. The projector 1 emits a stream of light l(fn) with a given frequency fn associated with a given command cmdₙ of a component of the device 2, for example with the command cmdₙ triggered by action on a physical button 21n of the electronic device 2.

In the example of FIG. 5a, the given command cmdₙ is a command to wake up the electronic device 2 or put it on standby depending on the “standby” or “active” status of the electronic device 2. The projector 1 is then placed relative to the electronic device 2, and more particularly to the physical activation/standby button 21n, such that the projector 1 projects the stream of light l(fn) onto a projection area zₙ placed on the electronic device 2: 26n.

Particularly, the projector 1 is fixed in place by an attachment 11 (not illustrated) to the electronic device 2. The attachment is in particular an adhesive-based attachment, such as a double-sided tape, a magnetism-based attachment, a clip-based attachment, etc.

For example, when the electronic device 2 is made of metal or covered by a metal sticker, the attachment comprises a magnet for attaching the projector 1 to the electronic device 2. In contrast, when the electronic device 2 has a magnetic external surface or is covered by a magnetic sticker, the attachment is made of metal. The magnet-based or metal-based attachment then allows magnetic bonding to the electronic device 2.

Optionally, the attachment is in particular a clip-based attachment such as a suction cup, notches, etc., allowing the projector 1 to be fitted onto the electronic device 2. In particular, an attachment base comprising fins or lugs is adhesively bonded to the electronic device 2, the fins/lugs being capable of being fitted into the notches of the attachment of the projector 1.

Particularly, the attachment 11 is composed of two separable parts:
a first part called the holder, which is able to hold the projector 1 in relation to the electronic device 2, and
a second part that is attachable to the holder, the actual projector 1 capable of emitting a stream of light l(fn) with a given frequency fn comprising the second part of the attachment 11.

FIG. 5b shows a simplified schematic of a second mode of use of a neural stimulator-projector according to the invention, in which the neural stimulator-projector projects onto a plurality of electronic devices placed in a room.

The projector 1 is a fixed projector, and optionally a 360° projector as illustrated in FIG. 5 b, placed on a fixed device of the room, in which is found at least one electronic device 2_{α}, 26_{β} 2_{γ} to which at least one neural-interaction function is added. In the example in FIG. 5b, the electronic device 2_{α} is a lamp, the electronic device 2_{β} is a television set, and the electronic device 2_{γ} is an HDD and/or DVD media player/recorder.

The fixed projector 1 in particular comprises an attachment 11 such as described in FIG. 5a.

The projector 1 diffuses at least one stream of light l(f_{α}), l(f_{β1}) . . . l(f_{βn}) and l(f_{γ1}) . . . l(f_{γ4}), respectively, with a given frequency, f_{α}, f_{β1} . . . f_{βn} and f_{γ1} . . . f_{γ4}, respectively, associated with a given command cmd_{α}, cmd_{β1} . . . cmd_{βn} and cmd_{γ1} . . . cmd_{γ4}, respectively, of a component of the device 2_{α}, 2_{β} and 2_{γ}, respectively, in a given projection area z_{α}, z_{β1} . . . z_{βn} and z_{γ1} . . . z_{γ4}, respectively, also named 26_{α} when it is placed on the electronic device 2_{α}, and 6_{β1} . . . 6_{βn} and 6_{γ1} . . . 6_{γ4} otherwise, respectively.

In the example in FIG. 5b, the given frequency f_{α}, f_{β1} . . . f_{βn} and f_{γ1} . . . f_{γ4}, respectively, of the stream of light l(f_{α}), l(f_{β1}) . . . l(f_{βn}) and l(f_{γ1}) . . . l(f_{γ4}), respectively, projected into the given projection area z_{α}, z_{β1} . . . z_{βn} and z_{γ1} . . . z_{γ4}, respectively, is associated with a given command cmd_{α} to switch on/off the lamp 2_{α}, with a given command cmd_{β1} to activate/place on standby the television set 2_{β}, with a given command cmd_{β2} to increase the volume of the television set 2_{β}, . . . , with a given command cmd_{βn} to switch the television set 2_{β} to the next channel, with a given command cmd_{γ1} to activate/place on standby the player/recorder 2_{γ}, with a given command cmd_{γ2} to rewind the player/recorder 2_{γ}, with a given command cmd_{γ3} to pause/play the player/recorder 2_{γ}, . . . , and with a given command cmd_{γ4} to fast forward the player/recorder 2_{γ}, respectively.

When the projector 1 is targeted, it emits the streams of light associated with one of the electronic devices 2_{α}, 2_{β}, 2_{γ} of the room, respectively l(f_{α}) associated with the second electronic device 2_{α}, l(f_{β1}) . . . l(f_{βn}) associated with the second electronic device 2_{β}, and l(f_{γ1}) . . . l(f_{γ4}) associated with the second electronic device 2_{γ}, or, successively, (by scanning) the streams of light associated with various electronic devices 2_{α}, 2_{β} 2_{γ} of the room, respectively l(f_{α}) associated with the second electronic device 2_{α}, l(f_{β1}) . . . l(f_{βn}) associated with the second electronic device 2₆₂ , and l(f_{65 1}) . . . l(f_{γ4}) associated with the second electronic device 2_{γ}.

Thus any user U equipped with a direct neural interface 4, such as a BCI headset, located in or moving through the room, will look at the projected stream of light l(f_{α}), l(f_{β1}) . . . l(f_{βn}), and l(f_{γ1}) . . . l(f_{γ4}), respectively, positionally associated with the electronic device 2_{α}, 2_{β} and 2_{γ}, respectively, or even with a component 24_{α}, or with a physical button of this electronic device.

The given frequency f_{α}, f_{β1} . . . f_{βn}, or f_{γ1} . . . f_{γ4}, respectively, of the stream of light being looked at l(f_{α}), l(f_{β1}) . . . l(f_{βn}), or l(f_{γ1}) . . . l(f_{γ4}), will trigger a visual evoked potential in the electroencephalogram signals, the frequency of which, called the neural frequency, will be detected by the direct neural interface 4. The neural frequency is dependent on the given frequency of the stream of light being looked at. The direct neural interface 4 transmits the detected neural frequency to at least one electronic device 2_{α}, 2_{β}, 2_{γ} such that the electronic device 2_{α}, 2_{β}, or 2_{γ}, respectively, a given command of which is associated with a given frequency of the stream of light being looked at is controlled by the given command associated with the given frequency on which the detected neural frequency is dependent.

Simply put, the user U by looking at a stream of light projecting a visual signal indicative of a given command near to/onto an electronic device controls, via neural interaction, implementation of the given command in the electronic device.

The advantage of such a fixed projector 1 scanning the room with streams of light either simultaneously (360° projector) or successively (rotary projector) is to allow a plurality of users equipped with direct neural interfaces present in the room to be able to interact with the same electronic device or a plurality of distinct electronic devices present in the room.

FIG. 5c shows a simplified schematic of a third mode of use of a neural stimulator-projector according to the invention, in which the neural stimulator-projector is attached to, or even integrated into, the direct neural interface worn by the user.

The projector 1, 41 is a nomadic projector worn by a user U equipped with a direct neural interface 4. Optionally, the direct neural interface 4 is capable of having a projector 1 attached to the direct neural interface 4 or comprises a projector 41. Thus, the projector 1, 41 allows the user U moving through a room or through a plurality of rooms to interact neurally not only with a plurality of electronic devices in the same room but also in different rooms, or even different buildings.

Thus, a moving user U passes, at a first time t1, in front of a first electronic device 2_{α}, in the present case a lamp, then sits down later, at a second time t2 different from the first time t1, in front of a second electronic device 2_{β}, in the present case a television set to which at least one neural-interaction function is added.

The projector 1, 41 emits at least one stream of light l(f_{α}) at the first time t1, and l(f_{β1}) . . . l(f_{βn}) at the second time t2, respectively, with a given frequency f_{α}, f_{β1} . . . f_{βn}, respectively, associated with a given command cmd_{α}, cmd_{β1} . . . cmd_{βn}, respectively, of a component of the device 2_{α}, 2_{β}, respectively, in a given projection area z_{α}, z_{β1} . . . z_{βn}, respectively, also denoted 26_{α} when it is placed on the electronic device 2_{α}, and 6_{β1} . . . 6_{βn} otherwise, respectively.

In the example in FIG. 5c, the given frequency f₆₀ , f_{β1} . . . f_{βn}, respectively, of the stream of light l(f_{α}), l(f_{β1}) . . . l(f_{βn}), respectively, projected into the given projection area z_{α}, z_{β1} . . . z_{βn}, respectively, is associated with a given command cmd_{α} to switch on/off the lamp 2_{α}, with a given command cmd_{62 1} to switch the on/off the television set 2_{β}, with a given command cmd_{β2} to increase the volume of the television set 2_{β}, . . . , and with a given command cmd_{βn} to switch the television set 2_{β} to the next channel.

Thus any user U equipped with a direct neural interface 4, such as a BCI headset, who finds her or himself facing the lamp 2_{α}at the first time t1 will see the projected stream of light l(f_{α}), and who finds her or himself facing the television set 2_{β} at the first time t2 will see a projected stream of light l(f_{β1}) . . . l(f_{βn}), respectively, which projected streams are positionally associated with the electronic device 2_{α}, 2_{β}, respectively, or even with a component 24_{α} or with a physical button of this electronic device.

The given frequency f_{α}, f_{β1} . . . f_{βn}, respectively, of the stream of light looked at l(f_{α}) l(f_{β1}) . . . l(f_{βn}) will trigger a visual evoked potential in the electroencephalogram signals the frequency of which, called the neural frequency, will be detected by the direct neural interface 4. The neural frequency is dependent on the given frequency of the stream of light being looked at. The direct neural interface 4 transmits the detected neural frequency to at least one electronic device 2_{α}, 2_{β}, such that the electronic device 2_{α}, 2_{β}, respectively, a given command of which is associated with a given frequency of the stream of light being looked at is controlled by the given command associated with the given frequency on which the detected neural frequency is dependent.

Simply put, at a first time t1, the user U passing in front of the lamp 2_{α}, by looking at a stream of light projecting a visual signal indicative of a given command near to/onto the lamp controls, via neural interaction, implementation of the given command by the lamp. Next, at a second time t2, the user U sitting in front of the television set 2_{β}, by looking at a stream of light projecting a visual signal indicative of a given command near to/onto the television set controls, via neural interaction, implementation of the given command by the television set.

The advantage of such a nomadic projector 1 is to provide a plurality of users equipped with direct neural interfaces present in a plurality of rooms in which the user equipped with the nomadic projector finds her or himself with the ability to interact with the same electronic device or a plurality of distinct electronic devices present in the room.

The invention also targets a medium. The information medium may be any entity or device capable of storing the program. For example, the medium may comprise a storage means, such as a ROM, for example a CD-ROM or a microelectronic circuit ROM, or else a magnetic storage means, for example a floppy disk or a hard disk.

Moreover, the information medium may be a transmissible medium such as an electrical or optical signal, which may be routed via an electrical or optical cable, by radio or by other means. The program according to the invention may in particular be downloaded from a network, and particularly from the Internet.

As an alternative, the information medium may be an integrated circuit into which the program is incorporated, the circuit being designed to execute or to be used in the execution of the method in question.

In another implementation, the invention is implemented by way of software and/or hardware components. With this in mind, the term module may correspond equally to a software component or to a hardware component. A software component corresponds to one or more computer programs, one or more subroutines of a program or, more generally, to any element of a program or of software that is able to implement a function or a set of functions in accordance with the above description. A hardware component corresponds to any element of a hardware assembly that is able to implement a function or a set of functions.

1. A projector of comprising:
an emitter of a stream of light with a given frequency and arranged to project the stream of light into a given projection area anchored to an electronic device that is connectable to a communication network,
the projector being connectable to a communication network, the given frequency being associated with a given command of the electronic device.

2. The projector as claimed in claim 1, wherein the given command is identical to a given command triggered by a physical button of the electronic device.

3. The projector as claimed in claim 1, wherein the projector comprises a recognizer of the electronic device, this recognizer delivering an electronic-device identifier depending on which the given frequency is determined.

4. The projector as claimed in claim 1, wherein the projector comprises a locator of the electronic device, the locator delivering an electronic-device position depending on which the given projection area is determined.

5. The projector as claimed in claim 1, wherein the projector emits a plurality of streams of light with distinct given frequencies.

6. The projector as claimed in claim 5, wherein the given frequencies are associated with given commands of a plurality of electronic devices, each stream of light being projected with a given frequency into the given projection area around the electronic device a given command of which is associated with the given frequency among predefined geographic areas around the electronic devices.

7. The projector as claimed in claim 1, wherein the projector is one of the following devices:
a component of a direct neural interface capable of sensing a user-related frequency during projection of the stream of light with the given frequency, the direct neural interface comprising a sensed-neural-frequency transmitter capable of triggering a given command of the electronic device, which given command is associated with the given frequency of the stream of light emitted by the projector, the transmitted neural frequency being dependent on the given frequency;
a fixed projector comprising an attachment to a fixed device relative to at least one electronic device;
a nomadic projector comprising an attachment to a user portable device.

8. A manager of objects connected to a communication network, the objects including at least a projector and an electronic device, wherein the manager of the connected objects comprises:
a coupler configured to associate a given frequency of a stream of light emitted by the projector into a given projection area anchored to the electronic device, which is connected to the communication network, with a given command of the electronic device connected to the communication network.

9. A manager of objects connected to a communication network, the object objects including at least a projector and an electronic device, wherein the manager of the connected objects comprises:
a controller configured to control, by a given command, the electronic device, which is connected to the communication network, depending on a neural frequency received from a direct neural interface during emission of a stream of light with a given frequency by the projector into a given projection area anchored to an electronic device connected to the communication network, the received neural frequency being dependent on the given frequency, the given frequency and the given command being associated.

10. The manager of connected objects as claimed in claim 8, wherein the manager of the connected objects further comprises:
a controller configured to control, by the given command, the electronic device depending on a neural frequency received from a direct neural interface during emission of the stream of light with the given frequency by the projector into the given projection area, the received neural frequency being dependent on the given frequency, the given frequency and the given command being associated.

11. A direct neural interface capable of sensing a user-related frequency during projection of a stream of light with a given frequency, wherein the direct neural interface comprises:
a sensed-neural-frequency transmitter that is configured to transmit a neural frequency via a communication network, the sensed-neural-frequency transmitter being configured to trigger a given command of an electronic device associated with the given frequency of a stream of light emitted by a projector into a given projection area anchored to the electronic device, which is connected to the communication network, the transmitted neural frequency being dependent on the given frequency.

12. An electronic device connected to a communication network, wherein the electronic device comprises:
an actuator configured to control a component of the electronic device depending on at least one given command, the given command being dependent on a neural frequency transmitted by a direct neural interface during emission of a stream of light with a given frequency by a projector into a given projection area anchored to the electronic device, which is connected to the communication network, the transmitted neural frequency being dependent on the given frequency, the given frequency and the given command being associated.

13. The electronic device as claimed in claim 12, wherein the electronic device and the projector are physically associated and the electronic device comprises a power supply for the projector among the following power supplies:
a power-supply connector capable of being connected to the projector;
an induction-based charger capable of recharging a battery of the projector.

14. A method of initialization of a projector and of an electronic device that is connected to a communication network, wherein the method comprises:
associating a given frequency of a stream of light emitted by a projector into a given projection area anchored to an electronic device connected to the communication network with a given command of the electronic device, which is connected to the communication network.

15. A method of controlling an electronic device, wherein the method comprises:
controlling, by a given command, an electronic device connected to a communication network depending on a neural frequency received from a direct neural interface during emission of a stream of light with a given frequency by a projector into a given projection area anchored to the electronic device, which is connected to the communication network, the received neural frequency being dependent on the given frequency, the given frequency and the given command being associated.

16. A non-transitory computer readable medium comprising program code instructions stored thereon for executing the method of initialization of a projector as claimed in claim 14 when said instructions are executed by a processor.

17. A non-transitory computer readable medium comprising program code instructions stored thereon for executing the method of controlling an electronic device as claimed in claim 15 when said instructions are executed by a processor.
